SEQUENCE LISTING (30 sequences)

SEQ ID NO: 1 - DNA, 40 bp, synthetic construct
  source 1..40: mol_type = other DNA
gcttataagcgcgacaaaggatgtgttcgttctatgccat

SEQ ID NO: 2 - DNA, 40 bp, synthetic construct
  source 1..40: mol_type = other DNA
ggatgtcggtgaagctgcgcccatttttttgacggcgttg

SEQ ID NO: 3 - DNA, 20 bp, synthetic construct
  source 1..20: mol_type = other DNA
tggcggatcttccggacgat

SEQ ID NO: 4 - DNA, 20 bp, synthetic construct
  source 1..20: mol_type = other DNA
gcgatcgcctgctgcaaggt

SEQ ID NO: 5 - DNA, 40 bp, synthetic construct
  source 1..40: mol_type = other DNA
caacgccgtcaaaaaaatgggcgcagcttcaccgacatcc

SEQ ID NO: 6 - DNA, 40 bp, synthetic construct; identical to SEQ ID NO: 2
  source 1..40: mol_type = other DNA

SEQ ID NO: 7 - DNA, 41 bp, synthetic construct
  source 1..41: mol_type = other DNA
cctagatcctttaattcgagccgcgatcagatgctgcttga

SEQ ID NO: 8 - DNA, 40 bp, synthetic construct; identical to SEQ ID NO: 2
  source 1..40: mol_type = other DNA

SEQ ID NO: 9 - DNA, 40 bp, synthetic construct
  source 1..40: mol_type = other DNA
tggtggtgtcgttgttggcatggatcgtcgcgcatcagac

SEQ ID NO: 10 - DNA, 41 bp, synthetic construct
  source 1..41: mol_type = other DNA
tcaaacatgagaatagcttgccccaggtgccgatatcgtcc

SEQ ID NO: 11 - DNA, 1001 bp, synthetic construct
  source 1..1001: mol_type = other DNA
tggcggatcttccggacgatcggcacgctgtatcgtatcgagcggccggtgctctatttcggcgggatcggcgccgtgctggtgctggcggcggtgatcctggcgctgccgctgctgttcacctggctccagaccgggctggttccccgctatccgaccgcggtgctggcgaccggccttaccatcgtcgccttcctcagtttcgcctgcggcctcatcctcgacacggtggtgcacgggcggcgcgagatgcggcggatcgcctatctttcgcatgctgcgccgggcgcggccgacgcccgaagcgaggccccttgaagccgccccgctttcacccgatgtagggacacggcaaaaggcttataagcgcgacaaaggatgtgttcgttctatgccatagtgcactgcaacacgcgattcaacgttggtcccggcacgcgtcgggatgcaacttcctgtcgtacgttcgtgctggcgcctgagccggttgaatgctgcgcgaggtcctgtcccacccaacagaggcagccagctacacgcatgaagaaactgatcggacgactctgccaaggcctcagcctggctctgctctgctcgatgtcgctgggcgcttgcagcaccggcccggagatggcgtcttcgctgccgcatccggacccgctggcaatgtccacggtgcagcccgaataccgtcttgcgccgggcgatctgttgctggtgaaggtgtttcagatcgacgatctggagcggcaggtccgcatcgaccagaacggtcacatctcactgccgttgattggcgacgtcaaggccgccggtctgggcgttggcgaactggaaaagctggtcgccgatcggtatcgcgcaggctacctgcagcagccgcagatttcggtattcgtgcaggagtccaacgggcgtcgcgtcacggtcactggtgcggtagacgagccgggcatctacccggtgatcggcgccaacctcaccttgcagcaggcgatcgc

SEQ ID NO: 12 - DNA, 240 bp, synthetic construct
  source 1..240: mol_type = other DNA
ctgctgttcacctggctccagaccgggctggttccccgctatccgaccgcggtgctggcgaccggccttaccatcgtcgccttcctcagtttcgcctgcggcctcatcctcgacacggtggtgcacgggcggcgcgagatgcggcggatcgcctatctttcgcatgctgcgccgggcgcggccgacgcccgaagcgaggccccttgaagccgccccgctttcacccgatgtagggacacg

SEQ ID NO: 13 - DNA, 1287 bp, synthetic construct
  source 1..1287: mol_type = other DNA
atggctacgaacgctctgaagaagatcggtggcgatgccgcgatggcggcgccatcgcacgctcagcccttctcgtccccgctgctcgataccaagccgctcgccggcaagcgggtgctgatcgtggtggaaaacctgccggtgccgttcgaccggcgcgtgtggcaggaagcgcgcacgctcaaggcggcgggcgcgcatgtctcgatcatctgccccaccggcaaggggcatgaggcgcggcgcgagacgctggacggcatcgagatctaccggcacccgctgccgctcgatgcctcgggcgcgatgggctatctcgccgaatatggcgccgcgctgttctgggaaacggtctacgcctggaagatcttcttcaagggcgggatcgacgtgatccacggctgcaacccgcccgacctgatctttctggtgtcgctgccgctgcgcctgttcggcgtgcgctacctgttcgaccatcatgacatcaacccggagctttacgaggcgaagttcgacaagcgcggcttcttctggaagctgatggtgctgttcgaaaagctcaccttcaaggcggcgcgcgtgtcgatcgccaccaacgaaagctatcgccgcatcgccatcgagcgtggcggcaagaagcccgaggatgtgttcgtcgtccgctcggggcccgatctcacccgcgtccgcgcggtgccggtcaacccggcgtggaagaaggggcggcgcttcatggtcggctatgtcggcgtgatgggcgaccaggagggcatcgacctgctgctcgaatccgcgcgccacatcgtctacgacaagaagcgcgacgacgtgcagttctgcctcgtcggcggtggccccagcctcaaggggctgcaggcgatggccgccgagatgggcctgtccgatcacgtcaccttcaccggccgcgcgcccgaccaggatctgttcgaggtgctctgcaccgcggacgtctgcgtgaaccccgatcgggtcaacccgatgaacgacaagtcgaccatgaacaagatcatggaatatatggcgatggagaaggccatcgttcagttcgacgtggccgagggcaagttcagcgcgcagcaggcctcgctctatgcgaaggcgaacgatccggtcgacatggccgaaaagatcctcggcctgctcgacgaccccgccaagcgcgccgaaatgggcgccttcggccgcaaccgcgtcgaaaaggaactggactggtcgcgccagatcgagccgctgatcgccgcgtaccagcgggtgctgcggaagtag

SEQ ID NO: 14 - DNA, 4512 bp, synthetic construct
  source 1..4512: mol_type = other DNA
atggcgacttcccccttccgcccgatcggccagccgctgccgcgccagcccgttcgcaccttcctcagttcggcgctgctgggcatcgcgctgatccttggcggcggcggcgcgcgcttccccgccaccgagatcgttgtgcagtgcacagccctgatcgtgctcgtgtacatgctcgcggaatatcgccgcgaccgggccgcgctgttcgactggctgggaatcggcttcgtgctgctggtgctgctgctgccgctggtgcaactgatcccgctgccgcccgcgacctggcgggcactgccgggccgcgatttcctgctgcaggccgccgacttcaccggcacgctcgaccagccgcgcccgctcagcctgattccggatgcgacggtgtcgatgtggctgaccctgatcgtgcccgtcacgatgttcgtcgccgtgctgcaggcggacggacgcgagatccgcttgctgctgtgggtcgtcgtcggtgcggcggttgccggcgcgttgctcgcgatggtgcaggcggtgttcggcgacagcgacgccgtctatttctacaacacctcgcagcagggattgcccaccggcgtgttcgccaaccgcaatcaccaggcattgctgatggtgctggcgctgctcacggcctggctgctcgcgcgcgaccggcggcgcaagggccgcgcgctggcgatacgatggggtctgttcgggctgatggcgctgttcgcagccgcagcattggccaccgcctcgcgcaccggctcggtgctgctgctgctcgcgctcgcggccgtgttcgtgccgatggtggtgcgccatcgccgccgctctgcgctgatcggcggcagcatcgccggggtgaccgttctgctggtcgcgatcctggtgcagaccggcgcgttccagcgcctgatgggccgcttcgcgctggatgacgacaaccgctttcacttctggcccgacgtgacctacgcgatcggctcggtgtggccgttcggctcgggatacggcacgttcgacccctatttccgctcgatcgagagcctcgattcgctgggcagccatttcatcaaccatgcccacaacgactatctggaactggcgctggaaggcggcctgcccgcggtggtggcgatggcgttgttcgcgctcttcctcctggtcgcagcatggcgcatgctgcggcatcggcagccggggcccgaaggcggccgcggctggatcgcgctggcagggatattcctcgcaatgttgcatagcctcgtcgattatccattgcgcaccttcgcagttgcagcattattcgctctgctctgtggcctgctggtgcgggcgctgcgcccggcttcgagcgcagaggaggatgaaatggcgtagaaagagcttgcgcgcggggctgtgtgagaattgcatcggccaccgttcacacttataccgcatctgcttccacgcggggtggccgatgcgggcggatggccggaagagccgattgacgctgcgggtacggtaaaggggaagagatgagcaggttcgttacagtgtccgtggcaatcacgctgctgacgttgggtgggtgcgccggcacgaaacgttcctcgctgccgcgcggaccggtggcctacagcaccatcaaggcatccgatgccccgaccgtcgtgaccgactatcgcatcgccgcgggcgacacgctgtcggtcatcgtgttccaggaacccgatctttcgatcaaggacgttcccgtggatgccggcggcagcctttccataccgctgatcggccgcgtgcaggcggccggcaagacgagcttcgagctggccgacgagatcgccaagtcgctcggccgcaactatctcgtttcgcccaaggtctcggtctacgtgaccaccgccaccccgggccaggtcgtcgtcgaaggctcggtcaccagccccggcgtgttcccgatccggccgcagaccaccctcatcgaagcgctcgcgctcgccaaggggccgacccaggtcgcccgtctcgacgaggtgatcgtgttccgcaccgtcgacgggcagcgcatgggcgcggtgttcgacatcgccgcgatccgccgcggcgatgcggtcgatccgcgcatcgaacccaacgatgtcgtcgtggtcggctattcgggcctccgccaagtctatcgggattttatctcggccgcgccgctgatcggcgtcttcacccgcttctgatgcgcacgcgcctcacaaccgctccgttcaaggtgatctgatgaacgactccagcttccccgatcgccgcgacggaaacggcggacacgccgtgcgcgcgcgcgatgatggctatcaggcctacgtcgccgactatgatgaacaggatcagggcttccgcctcaacatcggcgccctgatcggcatcctccgccgcaactggatcccggtggcggtgatcgtcgcgctcgcgctgatcgccggtctcgtcgcgaccctgctcgccaccaagatctatcgctcggcggcgagcgtgcagatcgagcagcagaccgccaagatcgtcgacacgcagatgctggagccggcgacatcgatcgccgacgccaaccgcttcctcaacacgcagctcgccatcctgcagagccgcggcctcgcgatccgcaccgcggaacggatgaagctgttcgacaacagcaacttcctggtcgcgatgggcgtgaaggtagatgccgcgcagaccaacacgcccgaagcgatccgtcagcgtcgcgagatggtgatcaacctgctgcgcaccaacaccagcgtcgaactgccgaccaactcgcgcgtcgcccagcttcgcttcgactcgcccgatccgcgcctttccgcgcaggtcgccaacggcatcgccgatgcctatgtgaccggcaacctcgagcgcaagttcgacacctcggcctacgcacgcgactatctggccggccagctcgccgaacagaagcagcggctcgaggacagcgagcgggcgatgatcgcctatgcccgcgaatccgatctggtgacggtgggcggcgccggtggcgccagcggcggcgattccaatggtggtgagggtgtatccaagccgcagtcgctgacctcggcggcgctgctcgatctcaacaccgcctacacggcgtcgcgtgccgagcgcatcaaggccgagcagcgctggcgcgccgcgcagcgggcgccgctgatgagcctgcccgaagtgatcgaaaacggcgcggtgcaggccctcgtcggccagcgcgcgaccctgcgcggcgagttgcaggagcagcgccgccgccacaaggccgatcaccccgaggtgatccagctcaacgcgcagatcgccgagcttgatgcgcagatcaatcgccaggccaccgacatccgggactcgatccgcgaggcctatcagctcaccgtcggccaggaacagcagttcgacaccaccgtcggccagctgcggtccaccgcgcttgaggaacagggccgccaggtccgcatgaacatcctgcagcgtgaggtcgacaccaaccgcgaactctacaacggccttctccagcgcttcaaggaactgagcgcgacggccggcgtcgaatcgaacaacgtctcgatcatcgaccgggccgagcccgcgttcagcccgatcaagccgaggccgctggtcaatatggcgatcgcgctcgcgatcggcctcggccttgccgcgctgttcgtcttcgcccgcgaaaatctcgacgacgcgatccgcacgcccgacgatgtccagcgcatgctcaacgtgcctgcgctgggcgccgtcccgctgctcaaggccggcgagaacccgttcgaggaaatcagcaacccgcgctcggcgatggcggaagcctatcagtcgatccgcacggcgctggaactctcgaccgcgcacggcgtgccgcgcagcctggtgttcacgtccagccgcccgtcggaaggcaagtcgacctcggccaccgccaccgcgcagaacctggcgcggaccggcaagcgcgcgctgctcgtcgatgccgatcttcgtctgccgacgctgcacaagttcctcgacctcaagaacaatgccggcttcgtcagcttgctgaccggccagaagacggtgatggacgtgcgccagccgaccggactggaaggcttcgacttcatctcgtcgggcccgctgccgcctgccccggcggaactgctcagcgaggcgtcgctcaaccgcttcctcaacgatgcgctggcgcattacgacgtggtggtgatcgacgccccgccggtgatgggcttcgccgacgcgccgctgatcgcggcgatcaccgagggcacggtgttcgtggtcgaggccgacgtggcgcaccgtggtgccgcacggaccgcgatccgccgcctggtcaccagccgcgccaatgtggtcggcgccatcctgaccaagttcgacgccccggcgctgggctattacaactacaactacagctacagctacgaatatggaaaccgcgccaacgcgggctga

SEQ ID NO: 15 - DNA, 33 bp, synthetic construct
  source 1..33: mol_type = other DNA
gagataagagctccccttgcggatcgtgattct

SEQ ID NO: 16 - DNA, 38 bp, synthetic construct
  source 1..38: mol_type = other DNA
actgaacgatggccttctccggcaccggcaggttttcc

SEQ ID NO: 17 - DNA, 38 bp, synthetic construct
  source 1..38: mol_type = other DNA
ggaaaacctgccggtgccggagaaggccatcgttcagt

SEQ ID NO: 18 - DNA, 32 bp, synthetic construct
  source 1..32: mol_type = other DNA
gctgtcctctagaccgcctatgcagatacgct

SEQ ID NO: 19 - DNA, 17 bp, synthetic construct
  source 1..17: mol_type = other DNA
ccaaccgcacgatcaaa

SEQ ID NO: 20 - DNA, 19 bp, synthetic construct
  source 1..19: mol_type = other DNA
gttcgccttcgcatagagc

SEQ ID NO: 21 - DNA, 35 bp, synthetic construct
  source 1..35: mol_type = other DNA
gagcctcttaattaacaacccgctgacgatctggt

SEQ ID NO: 22 - DNA, 37 bp, synthetic construct
  source 1..37: mol_type = other DNA
gaccggagcagaatggcgagcgacggcgatgcagttc

SEQ ID NO: 23 - DNA, 37 bp, synthetic construct
  source 1..37: mol_type = other DNA
gaactgcatcgccgtcgctcgccattctgctccggtc

SEQ ID NO: 24 - DNA, 32 bp, synthetic construct
  source 1..32: mol_type = other DNA
gcaatgatctagacgtgacccggccctatgac

SEQ ID NO: 25 - DNA, 19 bp, synthetic construct
  source 1..19: mol_type = other DNA
caatgcgcgccatcataag

SEQ ID NO: 26 - DNA, 20 bp, synthetic construct
  source 1..20: mol_type = other DNA
gagcgccgatccttaatcac

SEQ ID NO: 27 - DNA, 19 bp, synthetic construct
  source 1..19: mol_type = other DNA
gccgccgccttcacatatt

SEQ ID NO: 28 - DNA, 19 bp, synthetic construct
  source 1..19: mol_type = other DNA
acgcgatagcccgagagga

SEQ ID NO: 29 - DNA, 19 bp, synthetic construct
  source 1..19: mol_type = other DNA
gcgggcaggaatcttggtg

SEQ ID NO: 30 - DNA, 19 bp, synthetic construct
  source 1..19: mol_type = other DNA
ccgcaattgccgatctgga

(19) United States
(12) Patent Application Publication
(10) Pub. No.: US 2026/0098104 A1
(43) Pub. Date: Apr. 9, 2026

(54) ULTRA-HIGH MOLECULAR WEIGHT XANTHAN GUM AND ITS PRODUCTION STRAIN AND DETECTION MOLECULAR MARKER

(71) Applicant: Nankai University, Tianjin (CN)

(72) Inventors: Ting Ma, Tianjin (CN); Mengmeng Wu, Tianjin (CN); Zhuangzhuang Shi, Tianjin (CN); Guoqiang Li, Tianjin (CN); Yue Ming, Tianjin (CN)

(21) Appl. No.: 18/911,228

(22) Filed: Oct. 9, 2024

(51) Int. Cl. C08B 37/00; C12N 1/20

(52) U.S. Cl. CPC C08B 37/0033; C12N 1/205; C12N 2510/02

(57) ABSTRACT

An engineered strain that produces a xanthan gum is constructed by genetic engineering methods. Upon testing, it is found that the monosaccharide composition and repeating units of the xanthan gum are the same as those of an ordinary xanthan gum, but a molecular weight of the xanthan gum is greater than 2.0×10⁷ Da, which is higher than a molecular weight range (0.2×10⁷-2.0×10⁷ Da) of the ordinary xanthan gum reported in the literature. Therefore, the xanthan gum is considered an ultra-high molecular weight xanthan gum, expanding application fields of the xanthan gum. A molecular marker for detecting a producing strain or processed products of the ultra-high molecular weight xanthan gum can rapidly identify an engineered strain synthesizing the ultra-high molecular weight xanthan gum and their mildly processed products.

### CROSS-REFERENCE TO RELATED APPLICATION

This application claims priority to Chinese Patent Application No. 202310886163.3, filed Jul. 19, 2023, which is herein incorporated by reference in its entirety.

### TECHNICAL FIELD

The disclosure relates to the field of microbial product technologies, and more particularly to an ultra-high molecular weight xanthan gum and its production strain and a detection molecular marker.

### STATEMENT REGARDING SEQUENCE LISTING

The sequence listing associated with this application is provided in text format in lieu of a paper copy and is hereby incorporated by reference into the specification. The name of the XML file containing the sequence listing is 24097TBYX-USP1-MF-2024-0099-SL.xml. The XML file is 34,709 bytes; is created on Sep. 24, 2024; and is being submitted electronically via patent center.

### BACKGROUND

Microbial exopolysaccharides are a bio-gum synthesized by microorganisms, with diverse properties, biocompatibility, degradability, and sustainable production. The microbial exopolysaccharides have been widely used in more than twenty industries, such as food, daily chemicals, pharmaceuticals, environmental protection, papermaking, petroleum and building materials, etc., for hundreds of applications, and are a class of biotechnological products closely related to people's lives. Xanthan gum is a natural polysaccharide discovered in the 1950s by the United States Department of Agriculture's Northern Region Research Laboratory, and the Food and Drug Administration (FDA) approved xanthan gum as a food additive for use in the food industry in 1969, generally as a stabilizer and a thickener. The xanthan gum is the second microbial exopolysaccharide to achieve industrial-scale production. Due to its high viscosity, high stability, salt tolerance, and high-temperature resistance, the xanthan gum has a wide range of applications in the fields of food, cosmetics, pharmaceuticals and petroleum industry etc.

The xanthan gum is a heteropolysaccharide, which is a high molecular weight polymer polymerized from pentasaccharide repeating units. The repeating unit of the xanthan gum includes two glucoses, two mannoses, and a glucuronic acid, with a molar ratio of 2:2:1. The two glucoses form a main chain, similar to a structure of cellulose, connected by a β-1-4-glycosidic bond. A side chain structure consists of the two mannoses spaced by the glucuronic acid, and the glucuronic acid is connected to the inner and outer mannoses by a β-1-4-glycosidic bond and a β-1-2-glycosidic bond, respectively. This trisaccharide side chain is connected to the two glucoses of the main chain at intervals through β-1-3-glycosidic bonds. A molecular weight of the xanthan gum generally ranges from 2×10⁶ to 2×10⁷ Daltons (Da). The viscosity, stability, salt tolerance, and high-temperature resistance of the xanthan gum are closely related to the molecular weight of the xanthan gum. The larger the molecular weight, the greater the viscosity of the xanthan gum, the better the viscoelasticity of the xanthan gum, and the stronger the salt tolerance and high-temperature resistance of the xanthan gum. The low viscosity and poor salt and high-temperature resistances of an ordinary xanthan gum limit the application of the ordinary xanthan gum in certain special fields.

Current research shows that the molecular weight of the xanthan gum is influenced by a bacterial strain, a carbon source, a nitrogen source, a fermentation temperature, a fermentation method, and a carbon/nitrogen (C/N) ratio during a fermentation process. Changing the molecular weight of the xanthan gum in the related art is mainly achieved by altering fermentation conditions during production, which results in poor stability and limited improvement in the molecular weight.

### SUMMARY

In view of this, a purpose of the disclosure is to provide an ultra-high molecular weight xanthan gum, which has a same monosaccharide composition as an ordinary xanthan gum; however, a molecular weight of the ultra-high molecular weight xanthan gum is much higher than a molecular weight range of the ordinary xanthan gum reported in the related art.

The disclosure further provides an engineered strain T-XM for producing the ultra-high molecular weight xanthan gum and a construction method thereof. The engineered strain T-XM capable of producing the ultra-high molecular weight xanthan gum can be obtained by inserting xanthan gum synthesis-related genes into a NXdPE strain from which Sanxan gum synthesis-related genes have been knocked out.

The disclosure provides an ultra-high molecular weight xanthan gum. A structure of each repeating unit of the ultra-high molecular weight xanthan gum is same as a structure of each repeating unit of an ordinary xanthan gum, and a molecular weight of the ultra-high molecular weight xanthan gum is greater than 2.0×10⁷ Da.

In an embodiment, each repeating unit of the ordinary xanthan gum includes two glucoses, two mannoses, and a glucuronic acid; the structure of each repeating unit includes the two glucoses connected by a β-1-4-glycosidic bond to form a main chain, a side chain structure of each repeating unit includes the two mannoses spaced by the glucuronic acid, the glucuronic acid is connected to the two mannoses which are inside and outside the glucuronic acid by a β-1-4-glycosidic bond and a β-1-2-glycosidic bond respectively, and the side chain structure is connected to the glucose of the main chain spaced by a β-1-3-glycosidic bond.

In an embodiment, the molecular weight of the ultra-high molecular weight xanthan gum is in a range of 2.91×10⁷ Da to 14.3×10⁷ Da.

The disclosure provides an engineered strain T-XM of Sphingomonas sanxanigenens for producing the ultra-high molecular weight xanthan gum. The engineered strain T-XM of Sphingomonas sanxanigenens is a Sphingomonas sp. strain NXdP with a part of Sanxan gum synthesis-related genes in the Sphingomonas sp. strain NXdP replaced by a xanthan gum synthesis gene cluster from Xanthomonas campestris.

In an embodiment, the xanthan gum synthesis gene cluster is obtained by amplification using a pair of primers with deoxyribonucleic acids (DNA) of the Xanthomonas campestris as a template.

The pair of primers includes a forward primer with the nucleotide sequence as shown in SEQ ID NO: 1 and a reverse primer with the nucleotide sequence as shown in SEQ ID NO: 2.

In an embodiment, the xanthan gum synthesis gene cluster is activated and expressed by a P₉₁₆ promoter of the Sphingomonas sp. strain NXdP. The part of the Sanxan gum synthesis-related genes includes an open reading frame (orf)0831 gene and an orf0533-orf0536 gene.

In an embodiment, a preservation number of the engineered strain T-XM is China General Microbiological Culture Collection Center (CGMCC) No. 27299.

The disclosure provides a construction method of the engineered strain T-XM, including the following steps:
knocking out the part of the Sanxan gum synthesis-related genes in the Sphingomonas sp. strain NXdP to obtain a defective strain NXdPE; and
cloning a P₉₁₆ promoter fragment from the Sphingomonas sp. strain NXdP and the xanthan gum synthesis gene cluster from the Xanthomonas campestris strain into the defective strain NXdPE to obtain the engineered strain T-XM for producing the ultra-high molecular weight xanthan gum.

In an embodiment, the cloning a P₉₁₆ promoter fragment from the Sphingomonas sp. strain NXdP and the xanthan gum synthesis gene cluster from the Xanthomonas campestris strain into the defective strain NXdPE to obtain the engineered strain T-XM includes:
constructing a recombinant vector by combining the P₉₁₆ promoter fragment, the xanthan gum synthesis gene cluster, and upstream and downstream homologous arms of the Sphingomonas sp. strain NXdP, conjugatively transferring the recombinant vector to the defective strain NXdPE, and then performing single crossover and double crossover screening to obtain the engineered strain T-XM with double crossover.

The disclosure provides a detection molecular marker for the engineered strain T-XM, the ultra-high molecular weight xanthan gum or processed products thereof. Primers for amplifying the detection molecular marker include: a forward primer with the nucleotide sequence as shown in SEQ ID NO: 3 and a reverse primer with the nucleotide sequence as shown in SEQ ID NO: 4.

The xanthan gum synthesis gene cluster: GenBank: U22511.1.

The disclosure provides the ultra-high molecular weight xanthan gum. The structure of each repeating unit of the ultra-high molecular weight xanthan gum is same as the structure of each repeating unit of the ordinary xanthan gum. The molecular weight of the ultra-high molecular weight xanthan gum is greater than 2.0×10⁷ Da. The disclosure utilizes genetic engineering methods to construct a strain named Sphingomonas sanxanigenens, with a strain number of T-XM, and the engineered strain T-XM includes the xanthan gum synthesis gene cluster from Xanthomonas campestris replacing the part of the Sanxan gum synthesis-related genes in the Sphingomonas strain NXdP. After fermenting and cultivating the engineered strain T-XM, the monosaccharide composition of the produced polysaccharide is determined. The monosaccharide composition of the ultra-high molecular weight xanthan gum includes the glucoses, glucuronic acid, and mannoses, which is consistent with that of the ordinary xanthan gum. After determined by Fourier transform infrared spectrometer, the structure of the ultra-high molecular weight xanthan gum is consistent with that of the ordinary xanthan gum. The molecular weight of the produced polysaccharide is measured by size exclusion chromatography with multi-angle laser light scattering (SEC-MALLS), and results show that the weight-average molecular weight of the ultra-high molecular weight xanthan gum is in a range of 2.91×10⁷ to 14.3×10⁷ Da, far exceeding the molecular weight range (2.0×10⁶ to 2.0×10⁷ Da) of ordinary xanthan gum reported in the literature. Viscosity measurements and tests for salt and heat resistance indicate that the ultra-high molecular weight xanthan gum has good viscosity, high stability, and high salt and heat resistance, making the ultra-high molecular weight xanthan gum a suitable stabilizer and thickener with broad applications in food, cosmetics, pharmaceutical, and petroleum industries.

### BRIEF DESCRIPTION OF DRAWINGS

FIG. 1 illustrates monosaccharide compositions of ultra-high molecular weight products determined by a high performance liquid chromatography (HPLC) method.

FIG. 2 illustrates a result of determining an ultra-high molecular weight xanthan gum by SEC-MALLS.

FIG. 3 illustrates another result of determining an ultra-high molecular weight xanthan gum by SEC-MALLS.

FIG. 4 illustrates structural determination results of the ultra-high molecular weight xanthan gum prepared by the disclosure, an ordinary xanthan gum, and a Sanxan gum.

FIG. 5 illustrates viscosity measurement results of the ultra-high molecular weight xanthan gum and the ordinary xanthan gum.

### BIOLOGICAL PRESERVATION INFORMATION

The Sphingomonas sanxanigenens (i.e., the engineered strain T-XM) is preserved at CGMCC, with a preservation date of May 9, 2023. The address is No. 3, Courtyard 1, Beichen West Road, Chaoyang District, Beijing, Institute of Microbiology, Chinese Academy of Sciences, and the preservation number is CGMCC No. 27299.

### DETAILED DESCRIPTION OF EMBODIMENTS

The disclosure provides an ultra-high molecular weight xanthan gum. A structure of each repeating unit of the ultra-high molecular weight xanthan gum is same as a structure of each repeating unit of an ordinary xanthan gum, and a molecular weight of the ultra-high molecular weight xanthan gum is greater than 2.0×10⁷ Da.

In the disclosure, a molecular weight of the ordinary xanthan gum is in a range of 2.0×10⁶ to 2.0×10⁷ Da. Each repeating unit of the ordinary xanthan gum includes two glucoses, two mannoses, and a glucuronic acid. The structure of each repeating unit includes the two glucoses connected by a β-1-4-glycosidic bond to form a main chain, a side chain structure of each repeating unit includes the two mannoses spaced by the glucuronic acid, the glucuronic acid is connected to the two mannoses which are inside and outside the glucuronic acid by a β-1-4-glycosidic bond and a β-1-2-glycosidic bond, respectively, and the side chain structure is connected to the glucose of the main chain spaced by a β-1-3-glycosidic bond. In embodiments of the disclosure, monosaccharide composition analysis is first carried out. The results show that the monosaccharide composition of the ultra-high molecular weight xanthan gum includes glucose, glucuronic acid, and mannose, which is consistent with the ordinary xanthan gum. In addition, Fourier transform infrared spectroscopy analysis is performed on the ordinary xanthan gum and the ultra-high molecular weight xanthan gum. The results indicate that spectra of both are consistent, suggesting that the repeating units of the ultra-high molecular weight xanthan gum are same as those of the ordinary xanthan gum.

In the disclosure, the molecular weight of the ultra-high molecular weight xanthan gum is preferably greater than 2.0×10⁷ Da. In the embodiments of the disclosure, the molecular weight of the ultra-high molecular weight xanthan gum is determined by SEC-MALLS. The results indicate that the weight-average molecular weight of the ultra-high molecular weight xanthan gum is in a range of 2.91×10⁷ to 14.3×10⁷ Da, far exceeding the molecular weight range (2.0×10⁶ to 2.0×10⁷ Da) of the ordinary xanthan gum reported in the literature, and falls within a category of ultra-high molecular weight xanthan gum.

The disclosure provides an engineered strain T-XM of Sphingomonas sanxanigenens for producing the ultra-high molecular weight xanthan gum. The engineered strain T-XM of Sphingomonas sanxanigenens is a Sphingomonas sp. strain NXdP with a part of Sanxan gum synthesis-related genes in the Sphingomonas sp. strain NXdP replaced by a xanthan gum synthesis gene cluster from Xanthomonas campestris.

In the disclosure, the xanthan gum synthesis gene cluster is obtained by amplification using a pair of primers with DNA of the Xanthomonas campestris as a template. The pair of primers includes a forward primer with the nucleotide sequence as shown in SEQ ID NO: 1 (GCTTATAAGCGCGACAAAGGATGTGTTCGTTCTATGCCAT) and a reverse primer with the nucleotide sequence as shown in SEQ ID NO: 2 (GGATGTCGGTGAAGCTGCGCCCATTTTTTTGACGGCGTTG). The disclosure does not have any special restrictions on the Xanthomonas campestris, and any well-known strain commonly used in the field can be used, such as strains with the preservation numbers CGMCC No. 15155 or American Type Culture Collection (ATCC) 33913. The Xanthomonas campestris strain with the preservation number CGMCC No. 15155 is derived from soil and is recorded and disclosed in a Chinese patent with publication number CN109706110A. The Xanthomonas campestris strain with the preservation number ATCC 33913 can be purchased through commercial channels.

In the disclosure, the xanthan gum synthesis gene cluster is activated and expressed by a P₉₁₆ promoter of the Sphingomonas sp. strain NXdP. Primers for amplifying the P₉₁₆ promoter includes a forward primer with the nucleotide sequence as shown in SEQ ID NO: 5 (CAACGCCGTCAAAAAAATGGGCGCAGCTTCACCGACATCC) and a reverse primer with the nucleotide sequence as shown in SEQ ID NO: 6 (GGATGTCGGTGAAGCTGCGCCCATTTTTTTGACGGCGTTG). The nucleotide sequence of the P₉₁₆ promoter is as shown in SEQ ID NO: 12 (CTGCTGTTCACCTGGCTCCAGACCGGGCTGGTTCCCCGCTATCCGACCGCGGTGC TGGCGACCGGCCTTACCATCGTCGCCTTCCTCAGTTTCGCCTGCGGCCTCATCCTC GACACGGTGGTGCACGGGCGGCGCGAGATGCGGCGGATCGCCTATCTTTCGCATG CTGCGCCGGGCGCGGCCGACGCCCGAAGCGAGGCCCCTTGAAGCCGCCCCGCTTT CACCCGATGTAGGGACACG).

In the disclosure, the part of the Sanxan gum synthesis-related genes includes an orf0831 gene and an orf0533-orf0536 gene. The nucleotide sequence of the orf0831 gene is shown in SEQ ID NO: 13, and the nucleotide sequence of the orf0533-orf0536 gene is shown in SEQ ID NO: 14.

In the disclosure, the engineered strain T-XM is preserved in an authority designated by the China National Intellectual Property Administration, and a preservation number of the strain T-XM is CGMCC No. 27299.

The disclosure provides a construction method of the strain T-XM, including the following steps:
knocking out the part of the Sanxan gum synthesis-related genes in the Sphingomonas sp. strain NXdP to obtain a defective strain NXdPE; and
cloning a P₉₁₆ promoter fragment from the Sphingomonas sp. strain NXdP and the xanthan gum synthesis gene cluster from the Xanthomonas campestris strain into the defective strain NXdPE to obtain the engineered strain T-XM for producing the ultra-high molecular weight xanthan gum.

In the disclosure, the knocking out the part of the Sanxan gum synthesis-related genes in the Sphingomonas sp. strain NXdP includes: knocking out the orf0831 gene and the orf0533-orf0536 gene sequentially which are related to Sanxan gum synthesis in the Sphingomonas sp. strain NXdP. A knocking out method refers to a double-crossover homologous recombination method disclosed in a Chinese patent with application No. 201810737183.3 (publication No. CN108795969A).

In the disclosure, the cloning a P₉₁₆ promoter fragment from the Sphingomonas sp. strain NXdP and the xanthan gum synthesis gene cluster from the Xanthomonas campestris strain into the defective strain NXdPE to obtain the engineered strain T-XM includes:
constructing a recombinant vector by combining the P₉₁₆ promoter fragment, the xanthan gum synthesis gene cluster, and upstream and downstream homologous arms of the Sphingomonas sp. strain NXdP, conjugatively transferring the recombinant vector to the defective strain NXdPE, and then performing single crossover and double crossover screening to obtain the engineered strain T-XM with double crossover.

In the disclosure, the constructing a recombinant vector includes the following steps: cloning the upstream homologous arm fragment, the P₉₁₆ promoter fragment, the xanthan gum synthesis gene cluster, and the downstream homologous arm fragment sequentially to a backbone vector to obtain the recombinant vector. A pair of primers for amplifying the upstream homologous arm fragment includes a forward primer with the nucleotide sequence as shown in SEQ ID NO: 7 (CCTAGATCCTTTAATTCGAGCCGCGATCAGATGCTGCTTGA), and a reverse primer with the nucleotide sequence as shown in SEQ ID NO: 8 (GGATGTCGGTGAAGCTGCGCCCATTTTTTTGACGGCGTTG). A pair of primers for amplifying the downstream homologous arm fragment includes a forward primer with the nucleotide sequence as shown in SEQ ID NO: 9 (TGGTGGTGTCGTTGTTGGCATGGATCGTCGCGCATCAGAC), and a reverse primer with the nucleotide sequence as shown in SEQ ID NO: 10 (TCAAACATGAGAATAGCTTGCCCCAGGTGCCGATATCGTCC). The cloning is carried out by a high-fidelity (HiFi) DNA assembly master mix one-step method to achieve ligation into the backbone vector. The disclosure does not impose any special restrictions on a preparation method of the backbone vector, and any well-known backbone vector commonly used in the field can be used. In the embodiment of the disclosure, the backbone vector is a pLO3 vector.

In the disclosure, a method for transferring the recombinant vector to the defective strain NXdPE and performing single crossover and double crossover screening refers to a transferring method disclosed in the Chinese patent with application No. 201810737183.3 (publication No. CN108795969A).

In the disclosure, a method for producing the ultra-high molecular weight xanthan gum using the strain T-XM obtained by the construction method refers to a hydrogel preparation method disclosed in the Chinese patent with application No. 201810737183.3 (publication No. CN108795969A), including the following steps.
inoculating the engineered strain T-XM into a traditional plant-growing (TPG) liquid medium for cultivating with shaking for 20-26 hours (h) to obtain a culture broth, inoculating the culture broth into a seed medium for cultivating with shaking for 20-26 h to obtain a seed broth, inoculating the seed broth into a fermentation medium for fermenting for 68-75 h to obtain a fermentation broth, precipitating the fermentation broth with ethanol followed by separating to obtain a precipitate, and removing moisture from the precipitate to obtain the ultra-high molecular weight xanthan gum.

In the disclosure, a temperature of the cultivating with shaking is in a range of 28-35° C., more specifically in a range of 30-32° C. The fermentation medium includes: 30-70 grams per liter (g/L) of glucose, 0.5-2 g/L of soybean cake powder, 1-2 g/L of dipotassium hydrogen phosphate (K₂HPO₄), 0.1-1 g/L of magnesium sulphate (MgSO₄), and 1-2 g/L of sodium nitrate (NaNO₃). A fermentation temperature is in a range of 28-35° C., more specifically in a range of 30-32° C. A method for removing moisture from the precipitate includes: drying at 60-90° C. for 2-6 h, in a specific embodiment, drying at 70-85° C. for 4 h. After removing moisture from the precipitate, the precipitate is pulverized and sieved through an 80-mesh sieve to obtain the ultra-high molecular weight xanthan gum. The TPG liquid medium includes: 8-12 g/L glucose, 3-7 g/L of peptone, 1-5 g/L of yeast powder and 1-5 g/L of beef extract.

The disclosure provides a detection molecular marker for the engineered strain T-XM, the ultra-high molecular weight xanthan gum or processed products thereof. The nucleotide sequence of the detection molecular marker is as shown in SEQ ID NO: 11 (TGGCGGATCTTCCGGACGATCGGCACGCTGTATCGTATCGAGCGGCCGGTGCTCT ATTTCGGCGGGATCGGCGCCGTGCTGGTGCTGGCGGCGGTGATCCTGGCGCTGCC GCTGCTGTTCACCTGGCTCCAGACCGGGCTGGTTCCCCGCTATCCGACCGCGGTG CTGGCGACCGGCCTTACCATCGTCGCCTTCCTCAGTTTCGCCTGCGGCCTCATCCT CGACACGGTGGTGCACGGGCGGCGCGAGATGCGGCGGATCGCCTATCTTTCGCAT GCTGCGCCGGGCGCGGCCGACGCCCGAAGCGAGGCCCCTTGAAGCCGCCCCGCT TTCACCCGATGTAGGGACACGGCAAAAGGCTTATAAGCGCGACAAAGGATGTGT TCGTTCTATGCCATAGTGCACTGCAACACGCGATTCAACGTTGGTCCCGGCACGC GTCGGGATGCAACTTCCTGTCGTACGTTCGTGCTGGCGCCTGAGCCGGTTGAATG CTGCGCGAGGTCCTGTCCCACCCAACAGAGGCAGCCAGCTACACGCATGAAGAA ACTGATCGGACGACTCTGCCAAGGCCTCAGCCTGGCTCTGCTCTGCTCGATGTCG CTGGGCGCTTGCAGCACCGGCCCGGAGATGGCGTCTTCGCTGCCGCATCCGGACC CGCTGGCAATGTCCACGGTGCAGCCCGAATACCGTCTTGCGCCGGGCGATCTGTT GCTGGTGAAGGTGTTTCAGATCGACGATCTGGAGCGGCAGGTCCGCATCGACCAG AACGGTCACATCTCACTGCCGTTGATTGGCGACGTCAAGGCCGCCGGTCTGGGCG TTGGCGAACTGGAAAAGCTGGTCGCCGATCGGTATCGCGCAGGCTACCTGCAGCA GCCGCAGATTTCGGTATTCGTGCAGGAGTCCAACGGGCGTCGCGTCACGGTCACT GGTGCGGTAGACGAGCCGGGCATCTACCCGGTGATCGGCGCCAACCTCACCTTGC AGCAGGCGATCGC). Primers for amplifying the detection molecular marker includes: a forward primer with the nucleotide sequence as shown in SEQ ID NO: 3 (TGGCGGATCTTCCGGACGAT), and a reverse primer with the nucleotide sequence as shown in SEQ ID NO: 4 (GCGATCGCCTGCTGCAAGGT).

In the disclosure, the detection molecular marker is an essential gene for the synthesis of the ultra-high molecular weight xanthan gum, and without this sequence, the strain cannot synthesize extracellular polysaccharides. The detection molecular marker is applied in identifying strains that synthesize the ultra-high molecular weight xanthan gum or in the mid-light processing of all ultra-high molecular weight xanthan gum products.

In the disclosure, a detection method includes the following steps:
extracting genomic DNA as a template from a sample to be tested, and performing polymerase chain reaction (PCR) amplification with the primers for amplifying the detection molecular marker to obtain an amplified product, detecting the amplified product by electrophoresis and sequencing to obtain a result of 1001 base pairs (bp) in length, indicating that the sample to be tested is an ultra-high molecular weight xanthan gum-producing strain or a processed product of the ultra-high molecular weight xanthan gum.

A detailed description of the ultra-high molecular weight xanthan gum, its production strain, and the detection molecular marker of the disclosure is provided below in conjunction with embodiments, which cannot be understood as limiting the scope of protection of the disclosure.

### Embodiment 1

A construction method of a strain for producing an ultra-high molecular weight xanthan gum includes the following steps.

1. Primers and their Specific Sequences Involved in this Experiment are Shown in Table 1.

TABLE 1
primer sequences

| | |
|---|---|
| 0831su | 5′-gagataagagctccccttgoggatcgtgattct |
| | (the underline part is SacI |
| | restriction enzyme site, |
| | SEQ ID NO: 15) |
| 0831sl | 5′-actgaacgatggccttctccggcaccggcaggt |
| | tttcc (SEQ ID NO: 16) |
| 0831xu | 5′-ggaaaacctgccggtgccggagaaggccatcgt |
| | tcagt (SEQ ID NO: 17) |
| 0831xl | 5′-gctgtcctctagaccgcctatgcagatacgct |
| | (the underline part is XbaI restriction |
| | enzyme site, SEQ ID NO: 18) |
| 0831-1 | 5′-ccaaccgcacgatcaaa (SEQ ID NO: 19) |
| 0831-2 | 5′-gttcgccttcgcatagagc (SEQ ID NO: 20) |
| 0533-0536 | 5′-gagcctcttaattaacaacccgctgacgatctggt |
| su | (the underline part is PacI |
| | restriction enzyme site, SEQ ID NO: 21) |
| 0533-0536 | 5′-gaccggagcagaatggcgagcgacggcgatgcagtt |
| sl | c (SEQ ID NO: 22) |
| 0533-0536 | 5′-gaactgcatcgccgtcgctegccattctgctceggt |
| xu | c (SEQ ID NO: 23) |
| 0533-0536 | 5′-gcaatgatctagacgtgacccggccctatgac |
| xl | (the underline part is XbaI restriction |
| | enzyme site, SEQ ID NO: 24) |
| 0533-0536 1 | 5′-caatgcgcgccatcataag (SEQ ID NO: 25) |
| 0533-0536 2 | 5′-gagcgccgatccttaatcac (SEQ ID NO: 26) |
| 916gumUC1 | GCCGCCGCCTTCACATATT (upstream single |
| | crossover verification primer, |
| | SEQ ID NO: 27) |
| 916gumUC2 | ACGCGATAGCCCGAGAGGA (upstream single |
| | crossover verification primer, |
| | SEQ ID NO: 28) |
| 916gumDC1 | GCGGGCAGGAATCTTGGTG (downstream |
| | single crossover verification |
| | primer, SEQ ID NO: 29) |
| 916gumDC2 | CCGCAATTGCCGATCTGGA (downstream |
| | single crossover verification |
| | primer, SEQ ID NO: 30) |

### 2. Experiment Method

(1) a Construction Method of Astrain NXdPE of Sphingomonas sp. That Cannot Synthesize Sanxan Gum.

The orf0831 gene and the orf0533-orf0536 gene are knocked out sequentially from a strain NXdp according to a method in the Chinese patent with application No. 201810737183.3 (publication No. CN108795969A) to obtain the strain NXdPE which is deficient in the synthesis of Sanxan gum and polyhydroxybutyrate (PHB). Specific steps are as follows.

Target genes are inactivated by double crossover homologous recombination. A genome of the strain NXdP is extracted by using an extraction kit, and upstream and downstream homologous arms of the target genes are amplified using the genome of the strain NXdP as a template, with primers gene-su/gene-sl and gene-xu/gene-xl and PrimeSTAR® DNA polymerase (Takara Bio, Tokyo, Japan), respectively. A PCR amplification system includes 0.5 microliters (μL) of 10-50 nanograms per microliter (ng/μL) DNA template, 0.4 μL of each primer at 20 micromoles per liter (μM), 12.5 μL of PrimeSTAR® premix polymerase, 2 milliliters (mL) of dimethyl sulfoxide (DMSO), and water added up to 25 μL. PCR reaction conditions include: 98° C. for 15 seconds (s), 55° C. for 10 s, 72° C. for 30 s, 35 cycles.

Upstream and downstream DNA fragments are connected by overlap PCR, and then detected by electrophoresis, and a target gene band is purified and recovered by using a gel recovery kit to obtain a recombinant fragment. The recombinant fragment and the pLO3 plasmid are simultaneously digested with restriction enzymes SacI and XbaI or PacI at 37° C. for 90 minutes (min) to obtain digested fragments, and the digested fragments are purified and recovered by a PCR purification kit to obtain recovered products. The recovered products of both are ligated overnight at 16° C. using T4 DNA ligase to obtain a recombinant plasmid pLO3-Agene, and the recombinant plasmid pLO3-Agene is transferred into E. coli S17 competent cells for amplification of the recombinant plasmid. A correct single colony with PCR detection (primers gene-su/gene-xl) is picked for glycerol preservation.

The knockout plasmid is introduced into the strain NXdP by conjugative transfer, and single-crossover and double-crossover screening are performed. A single-crossover strain has both chloramphenicol and tetracycline resistance, and the double-crossover is used to verify the correct single colony by PCR detection (detection primers for the corresponding gene 1/corresponding gene 2). The orf0831gene and the orf0533-orf0536 gene are sequentially knocked out.

PCR conditions include: a PCR amplification system including 0.5 μL of DNA template at 10-50 ng/μL, 0.4 μL of each primer at 20 μM, 12.5 μL of rTaq polymerase, 2 μL of DMSO, and water added up to 25 μL.

PCR reaction conditions include: pre-denaturation at 95° C. for 10 min; 94° C. for 45 s, 55° C. for 45 s, 72° C. for 60 s, for 35 cycles; final extension at 72° C. for 10 min.

(2) A construction method of a recombinant vector containing a xanthan gum synthesis gene cluster includes following steps S1-S5.

S1, genomes are extracted from a strain NXdP (CGMCC NO. 15406) and Xanthomonas campestris (CGMCC NO. 15155) separately.

S2, the xanthan gum synthesis gene cluster, a promoter fragment, and upstream and downstream homologous arm fragments are amplified to obtain PCR products.

A PCR amplification system includes 0.5 μL of DNA template at 10-50 ng/μL, 0.4 μL of each primer at 20 M, 12.5 μL of PrimeSTAR® premix polymerase, 2 mL of DMSO, and water added up to 25 μL.

Primers for the upstream homologous arm fragment are U-F (CCTAGATCCTTTAATTCGAGCCGCGATCAGATGCTGCTTGA, SEQ ID NO: 7)/U-R (GGATGTCGGTGAAGCTGCGCCCATTTTTTTGACGGCGTTG, SEQ ID NO: 8). The DNA template for the upstream homologous arm fragment is the genome of the strain NXdP.

Primers for the P₉₁₆ promoter are P916-F (CAACGCCGTCAAAAAAATGGGCGCAGCTTCACCGACATCC, SEQ ID NO: 5)/P916-R (GGATGTCGGTGAAGCTGCGCCCATTTTTTTGACGGCGTTG, SEQ ID NO: 6). The DNA template for the P₉₁₆ promoter is the genome of the strain NXdP.

Primers for the xanthan gum synthesis gene cluster are gum-F (GCTTATAAGCGCGACAAAGGATGTGTTCGTTCTATGCCAT, SEQ ID NO: 1)/gum-R (GGATGTCGGTGAAGCTGCGCCCATTTTTTTGACGGCGTTG, SEQ ID NO: 2). The DNA template for the xanthan gum synthesis gene cluster is the genome of the Xanthomonas campestris (CGMCC NO. 15155).

Primers for the downstream homologous arm fragment are D-F (TGGTGGTGTCGTTGTTGGCATGGATCGTCGCGCATCAGAC, SEQ ID NO: 9)/D-R (TCAAACATGAGAATAGCTTGCCCCAGGTGCCGATATCGTCC, SEQ ID NO: 10). The DNA template for the downstream homologous arm fragment is the genome of the strain NXdP.

PCR reaction conditions include: pre-denaturation at 98° C. for 30 s; 98° C. for 15 s, 54° C. for 15 s, 72° C. for 10-60 s, for 35 cycles; final extension at 72° C. for 10 min.

S3, the PCR products are separated by agarose gel electrophoresis, and a target band is recovered by gel to obtain a target fragment.

S4, the recombinant vector is constructed as follows.

The upstream homologous arm, the P₉₁₆ promoter, the xanthan gum synthesis gene cluster, and the downstream homologous arm are ligated sequentially to the pLO3 vector by a HiFi DNA Assembly Master mix one-step method, and the recombinant vector (pLO3-P916gum) is constructed.

S5, the target fragment is inserted into the genome of Sphingomonas sp. NXdPE.

According to a method in the Chinese patent with application No. 201810737183.3 (publication No. CN108795969A), the recombinant vector is conjugatively transferred into the acceptor strain NXdPE followed by single crossover and double crossover screening. Primers for detecting the single crossover include 916gumUC1, 916gumUC2, 916gumDC1, and 916gumDC2. A strain in which both upstream and downstream homologous arms have undergone single crossover is considered a double crossover strain. A strain obtained after double crossover screening is an engineered bacterial strain for producing the ultra-high molecular weight xanthan gum.

### Embodiment 2

A method for producing the ultra-high molecular weight xanthan gum by the strain T-XM includes the following steps.

According to a Chinese patent with application No. 201510110078.3 (publication No. CN104651284A) and the Chinese patent with application No. 201810737183.3 (publication No. CN108795969A), the strain T-XM is fermented to obtain a fermentation broth, the fermentation broth is added with 2-3 times the volume of ethanol followed by stirring to obtain a flocculent precipitate, a precipitate is collected from the flocculent precipitate and then dried at 60-90° C. for 2-6 h to obtain a dried precipitate, and the dried precipitate is pulverized and sieved through an 80-mesh sieve to obtain the ultra-high molecular weight xanthan gum.

### Embodiment 3

A monosaccharide composition of the ultra-high molecular weight xanthan gum is determined by sample acid hydrolysis and HPLC analysis in two steps.

1. Sample acid hydrolysis: 5 milligrams (mg) of a sample which is dried is weighed and added into an ampoule, and then added with 1 mL of trifluoroacetic acid at 2 moles per liter (M) followed by sealing the ampoule to obtain a sealed ampoule, and the sealed ampoule is placed in a blast drying oven with a set temperature of 120° C. for the sample acid hydrolysis for 10 h to obtain an acid lysate. During the sample acid hydrolysis, the sample in the sealed ampoule is mixed every 2 h. After sample acid hydrolysis, 500 μL of the acid lysate is taken and added to a 1.5 mL eppendorf (EP) tube followed by evaporating liquid in a water bath at 95° C., and if necessary, drying with nitrogen gas. Then, 200 μL of ultrapure water is added to the EP tube followed by adjusting the acid lysate to neutrality with 0.2 M sodium hydroxide to obtain a neutral solution. The neutral solution is centrifuged at 12000 gravitational accelerations (g) to remove insoluble matter and then filtered by a 0.22 micrometers (m) filter membrane.

2. 1 g/L standard sugar solutions (glucose, glucuronic acid, mannose) are prepared, and then filtered through a 0.22 m filter membrane.

3. Liquid chromatography conditions: an Agilent 1100 liquid chromatography system (Agilent Technologies, Inc.) equipped with a Waters Sugar-Pak™ Icolumn, and a mobile phase of 50 milligrams per liter (mg/L) ethylenediaminetetraacetic acid calcium disodium salt (EDTA-2NaCa) solution.

Chromatographic conditions: an injection volume of 20 μL, a column temperature at 85° C., a flow rate set at 0.5 milliliters per minute (mL/min), a differential refractometer (also referred to as refractive index detector or differential refractive index detector), and a detector temperature set at 35° C.

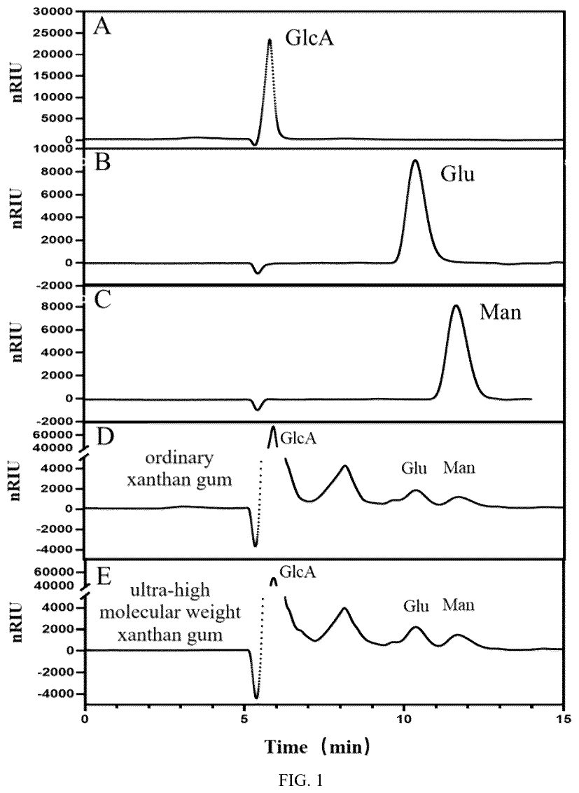

Results are shown as FIG. 1, indicating that the monosaccharide composition of the ultra-high molecular weight xanthan gum includes glucose, glucuronic acid, and mannose, which is consistent with that of an ordinary xanthan gum.

### Embodiment 4

### Determination of a Molecular Weight of a Product 1 by SEC-MALLS

The molecular weight of the product 1 is determined by SEC-MALLS according to methods reported in the literature.

Sample preparation: 10 mg of a dried sample is weighed and dissolved completely in 10 mL of ultrapure water, and then filtered through a 0.22 μm filter membrane.

Size exclusion chromatography conditions: an Agilent 1260 liquid chromatography system equipped with a Waters Ultrahydrogel™ Linear column, a mobile phase of a phosphate buffer (pH 7.2), an injection volume of 200 μL; a column temperature of room temperature, a flow rate set at 0.6 mL/min, and a differential refractometer with a detector temperature at 35° C.

Multi-angle laser light scattering instrument: MALLS, Wyatt Technology DAWN HELEOS, Santa Barbara, CA, USA.

Standard sample: Bovine serum albumin (BSA)

Data acquisition and analysis software: ASTRA™ software (Wyatt Technology).

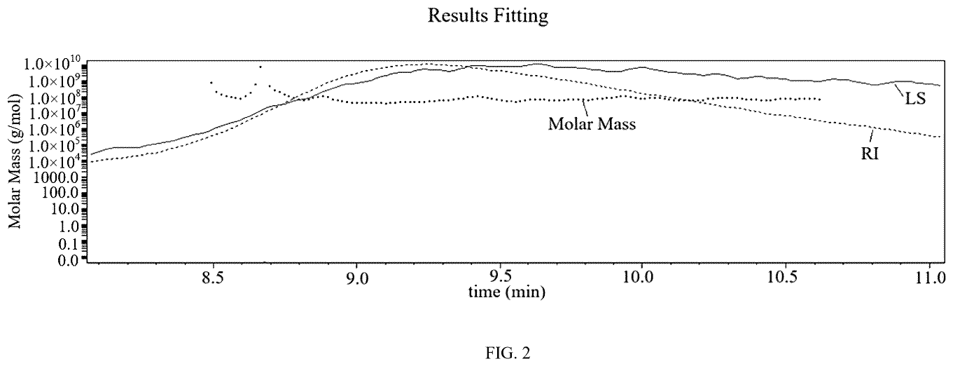

Results are shown as FIG. 2, indicating that a weight-average molecular weight of the ultra-high molecular weight xanthan gum is 2.91×10⁷ Da, which is far beyond a molecular weight range (2.0×10⁶-2.0×10⁷ Da) of the ordinary xanthan gum reported in the literature, and belongs to a category of ultra-high molecular weight xanthan gum.

Meanwhile, this method can be used to distinguish between ordinary xanthan gum products on the market and ultra-high molecular weight xanthan gum products.

### Embodiment 5

### Determination of a Molecular Weight of a Product 2 by SEC-MALLS

The molecular weight of the product 2 is determined by SEC-MALLS according to methods reported in the literature.

Sample preparation: 10 mg of a dried sample is weighed and dissolved completely in 10 mL of ultrapure water, and then filtered through a 0.22 m filter membrane.

Size exclusion chromatography conditions: an Agilent 1260 liquid chromatography system equipped with a Waters Ultrahydrogel™ Linear column, a mobile phase of a phosphate buffer (pH 7.2), an injection volume of 200 μL; a column temperature of room temperature, a flow rate set at 0.6 mL/min, and a differential refractometer with a detector temperature at 35° C.

Multi-angle laser light scattering instrument: MALLS, Wyatt Technology DAWN HELEOS, Santa Barbara, CA, USA.

Standard sample: BSA

Data acquisition and analysis software: ASTRA™ software (Wyatt Technology).

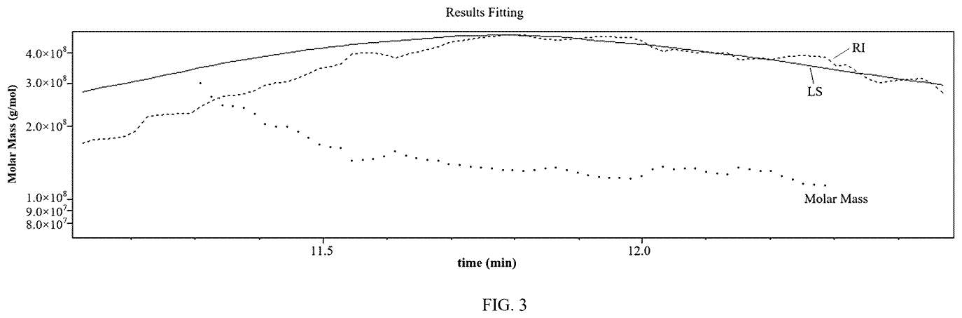

Results are shown as FIG. 3, indicating that a weight-average molecular weight of the ultra-high molecular weight xanthan gum is 1.43×10⁸ Da, which is far beyond a molecular weight range (2.0×10⁶-2.0×10⁷ Da) of the ordinary xanthan gum reported in the literature, and belongs to a category of ultra-high molecular weight xanthan gum.

Meanwhile, this method can be used to distinguish between ordinary xanthan gum products on the market and ultra-high molecular weight xanthan gum products.

### Embodiment 6

Primary structural differences between the ultra-high molecular weight xanthan gum, the ordinary xanthan gum, and the original polysaccharide Sanxan gum are identified by Fourier-transform infrared spectroscopy.

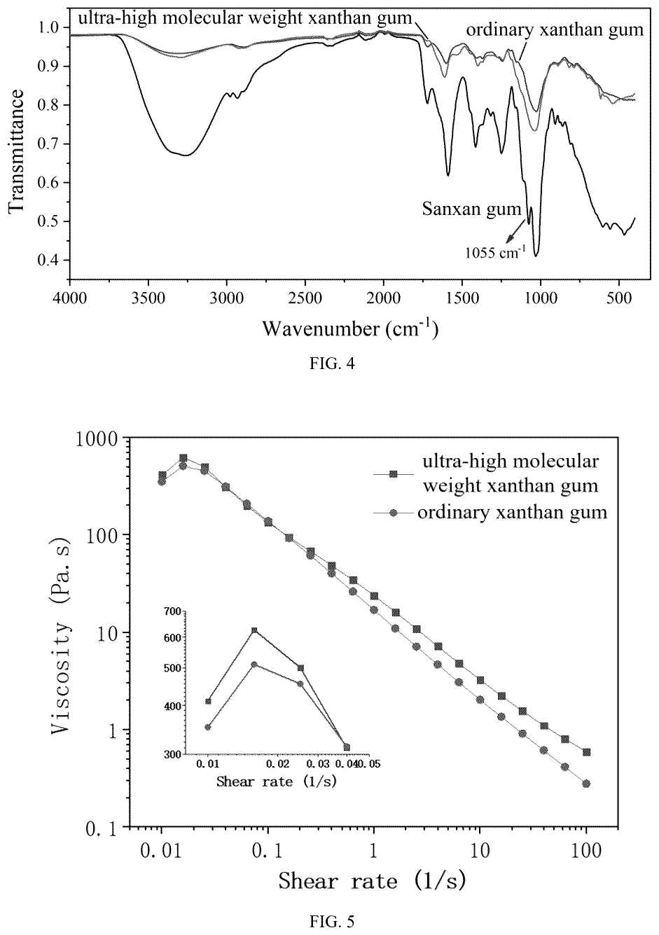

5 mg of a sample is ground and pressed with potassium bromide (KBr) for infrared scanning using a Nicolet 170SX infrared spectrometer. The presence or absence of certain functional groups is determined by the presence or absence of peaks at specific wavenumbers in the spectrum, and the strength of the peaks indicates the quantity, and the width and shape of the peaks are used to distinguish between various different functional groups. Specific results are shown in FIG. 4. The ultra-high molecular weight xanthan gum and the ordinary xanthan gum have essentially the same peak pattern, while the original polysaccharide Sanxan gum has a characteristic peak of L-rhamnose at 1055 reciprocal centimeters (cm⁻¹), indicating that the polysaccharide produced by the strain T-XM is the xanthan gum. The ultra-high molecular weight xanthan gum has functional groups that are essentially consistent in type and quantity with the ordinary xanthan gum, and thus it can be concluded that the ultra-high molecular weight xanthan gum and the ordinary xanthan gum have a structural consistency apart from the difference in molecular weight.

FIG. 4 illustrates infrared spectra of the ultra-high molecular weight xanthan gum, the ordinary xanthan gum, and the Sanxan gum (the original polysaccharide Sanxan gum).

### Embodiment 7

### Viscosity Determination of the Ultra-High Molecular Weight Xanthan Gum.

An ultra-high molecular weight xanthan gum solution at 1.0% is prepared. An ordinary xanthan gum synthesized by a preserved strain CGMCC NO. 15155 is used as a control. Viscosity changes under conditions of 0.01-1001 inverse seconds (s⁻¹) are measured by a TA DHR-1 rheometer.

Results are shown in FIG. 5. At extremely low shear rates, the viscosity of the ultra-high molecular weight xanthan gum is 626 Pascal seconds (Pa-s), while the viscosity of the ordinary xanthan gum is 510 Pa-s. As the shear rate increases, the difference in viscosity between the ultra-high molecular weight xanthan gum and the ordinary xanthan gum gradually increases. It is indicated that the ultra-high molecular weight xanthan gum has a higher viscosity than the ordinary xanthan gum.

### Embodiment 8

A molecular marker (the detection molecular marker) and an identification method for the ultra-high molecular weight xanthan gum-producing bacterial strain and its products.

Based on a gene sequence inserted into the genome of the strain NXdPE during the construction process of the strain T-XM, a portion of the gene sequence serves as the molecular marker with the nucleotide sequence shown as SEQ ID NO: 11 (TGGCGGATCTTCCGGACGATCGGCACGCTGTATCGTATCGAGCGGCCGGTGCTCT ATTTCGGCGGGATCGGCGCCGTGCTGGTGCTGGCGGCGGTGATCCTGGCGCTGCC GCTGCTGTTCACCTGGCTCCAGACCGGGCTGGTTCCCCGCTATCCGACCGCGGTG CTGGCGACCGGCCTTACCATCGTCGCCTTCCTCAGTTTCGCCTGCGGCCTCATCCT CGACACGGTGGTGCACGGGCGGCGCGAGATGCGGCGGATCGCCTATCTTTCGCAT GCTGCGCCGGGCGCGGCCGACGCCCGAAGCGAGGCCCCTTGAAGCCGCCCCGCT TTCACCCGATGTAGGGACACGGCAAAAGGCTTATAAGCGCGACAAAGGATGTGT TCGTTCTATGCCATAGTGCACTGCAACACGCGATTCAACGTTGGTCCCGGCACGC GTCGGGATGCAACTTCCTGTCGTACGTTCGTGCTGGCGCCTGAGCCGGTTGAATG CTGCGCGAGGTCCTGTCCCACCCAACAGAGGCAGCCAGCTACACGCATGAAGAA ACTGATCGGACGACTCTGCCAAGGCCTCAGCCTGGCTCTGCTCTGCTCGATGTCG CTGGGCGCTTGCAGCACCGGCCCGGAGATGGCGTCTTCGCTGCCGCATCCGGACC CGCTGGCAATGTCCACGGTGCAGCCCGAATACCGTCTTGCGCCGGGCGATCTGTT GCTGGTGAAGGTGTTTCAGATCGACGATCTGGAGCGGCAGGTCCGCATCGACCAG AACGGTCACATCTCACTGCCGTTGATTGGCGACGTCAAGGCCGCCGGTCTGGGCG TTGGCGAACTGGAAAAGCTGGTCGCCGATCGGTATCGCGCAGGCTACCTGCAGCA GCCGCAGATTTCGGTATTCGTGCAGGAGTCCAACGGGCGTCGCGTCACGGTCACT GGTGCGGTAGACGAGCCGGGCATCTACCCGGTGATCGGCGCCAACCTCACCTTGC AGCAGGCGATCGC), and a total length of 1001 bp. The upstream and downstream identification primers are sequences TXM1 (SEQ ID NO: 3: TGGCGGATCTTCCGGACGAT) and TXM2 (SEQ ID NO: 4: GCGATCGCCTGCTGCAAGGT). The molecular marker is used for identification, and the specific identification method includes the following steps (1)-(4).

(1) A genome of a tested sample such as a strain to be tested or a lightly processed product (e.g., food, daily chemical and petroleum-grade products containing xanthan gum) is extracted as a PCR template.

(2) A PCR amplification system includes: 10-50 ng/L DNA template at 0.5 μL, 20 M primer pair (TXM1/TXM2) at 0.4 μL, premix Taq polymerase at 12.5 μL, DMSO at 2 mL, and water added up to 25 μL.

(3) PCR reaction conditions include: pre-denaturation at 95° C. for 10 min; 94° C. for 45 s, 55° C. for 45 s, 72° C. for 60 s, for 35 cycles; final extension at 72° C. for 10 min.

(4) Electrophoresis detection is performed and a target fragment is recovered by gel. The target fragment is sent to a sequencing company for sequencing. A full length is 1001 bp and a sequencing result is consistent with the expected sequence, i.e., the tested sample is confirmed to be an ultra-high molecular weight xanthan gum-producing strain or a processed product of ultra-high molecular weight xanthan gum.

The above is only preferred embodiments of the disclosure. It should be pointed out that for those skilled in the art, several improvements and embellishments can be made without departing from the principles of the disclosure, and these improvements and embellishments should also be considered as the scope of protection of the disclosure.

What is claimed is:

1. An ultra-high molecular weight xanthan gum, wherein a structure of each repeating unit of the ultra-high molecular weight xanthan gum is same as a structure of each repeating unit of an ordinary xanthan gum, and a molecular weight of the ultra-high molecular weight xanthan gum is greater than 2.0×10⁷ Daltons (Da).

2. The ultra-high molecular weight xanthan gum as claimed in claim 1, wherein each repeating unit of the ordinary xanthan gum comprises two glucoses, two mannoses, and a glucuronic acid; the structure of each repeating unit comprises the two glucoses connected by a β-1-4-glycosidic bond to form a main chain, a side chain structure of each repeating unit comprises the two mannoses spaced by the glucuronic acid, the glucuronic acid is connected to the two mannoses which are inside and outside the glucuronic acid by a β-1-4-glycosidic bond and a β-1-2-glycosidic bond respectively, and the side chain structure is connected to the glucose of the main chain spaced by a β-1-3-glycosidic bond.

3. The ultra-high molecular weight xanthan gum as claimed in claim 1, wherein the molecular weight of the ultra-high molecular weight xanthan gum is in a range of 2.91×10⁷ Da to 14.3×10⁷ Da.

4. An engineered strain T-XM of Sphingomonas sanxanigenens for producing the ultra-high molecular weight xanthan gum as claimed in claim 1, wherein the engineered strain T-XM of Sphingomonas sanxanigenens is a Sphingomonas sp. strain NXdP with a part of Sanxan gum synthesis-related genes in the Sphingomonas sp. strain NXdP replaced by a xanthan gum synthesis gene cluster from Xanthomonas campestris.

5. The engineered strain T-XM as claimed in claim 4, wherein the xanthan gum synthesis gene cluster is obtained by amplification using a pair of primers with deoxyribonucleic acids (DNA) of the Xanthomonas campestris as a template; and
the pair of primers comprises a forward primer with the nucleotide sequence as shown in SEQ ID NO: 1 and a reverse primer with the nucleotide sequence as shown in SEQ ID NO: 2.

6. The engineered strain T-XM as claimed in claim 4, wherein the xanthan gum synthesis gene cluster is activated and expressed by a P₉₁₆ promoter of the Sphingomonas sp. strain NXdP; and
the part of the Sanxan gum synthesis-related genes comprises an open reading frame (orf)0831 gene and an orf0533-orf0536 gene.

7. The engineered strain T-XM as claimed in claim 4, wherein a preservation number of the strain T-XM is China General Microbiological Culture Collection Center (CGMCC) No. 27299.

8. A construction method of the engineered strain T-XM as claimed in claim 4, comprising:
knocking out the part of the Sanxan gum synthesis-related genes in the Sphingomonas sp. strain NXdP to obtain a defective strain NXdPE; and
cloning a P₉₁₆ promoter fragment from the Sphingomonas sp. strain NXdP and the xanthan gum synthesis gene cluster from the Xanthomonas campestris strain into the defective strain NXdPE to obtain the engineered strain T-XM for producing the ultra-high molecular weight xanthan gum.

9. The construction method of the engineered strain T-XM as claimed in claim 8, wherein the cloning a P₉₁₆ promoter fragment from the Sphingomonas sp. strain NXdP and the xanthan gum synthesis gene cluster from the Xanthomonas campestris strain into the defective strain NXdPE to obtain the engineered strain T-XM comprises:
constructing a recombinant vector by combining the P₉₁₆ promoter fragment, the xanthan gum synthesis gene cluster, and upstream and downstream homologous arms of the Sphingomonas sp. strain NXdP, conjugatively transferring the recombinant vector to the defective strain NXdPE, and then performing single crossover and double crossover screening to obtain the engineered strain T-XM with double crossover.

10. A detection molecular marker for the ultra-high molecular weight xanthan gum as claimed in claim 1 or processed products thereof, wherein primers for amplifying the detection molecular marker comprise: a forward primer with the nucleotide sequence as shown in SEQ ID NO: 3 and a reverse primer with the nucleotide sequence as shown in SEQ ID NO: 4.

11. A detection molecular marker for the engineered strain T-XM as claimed in claim 4, wherein primers for amplifying the detection molecular marker comprise: a forward primer with the nucleotide sequence as shown in SEQ ID NO: 3 and a reverse primer with the nucleotide sequence as shown in SEQ ID NO: 4.
